(19) United States
(12) Patent Application Publication
(10) Pub. No.: US 2022/0125777 A1
(43) Pub. Date: Apr. 28, 2022

(54) COMBINATION OF A CDK INHIBITOR AND A PIM INHIBITOR

(71) Applicant: Pfizer Inc., New York, NY (US)

(72) Inventors: Zhou Zhu, San Diego, CA (US); Yuan Liu, San Diego, CA (US)

(73) Assignee: Pfizer Inc., New York, NY (US)

(21) Appl. No.: 17/427,015

(22) PCT Filed: Jan. 31, 2020
(86) PCT No.: PCT/IB2020/050768

§ 371 Date: Jul. 29, 2021

Related U.S. Application Data
(60) Provisional application No. 62/964,484, filed on Jan. 22, 2020.
(60) Provisional application No. 62/799,746, filed on Feb. 1, 2019.

(51) Int. Cl. A61K 31/4545; A61K 31/4196; A61K 31/566; A61K 31/565; A61K 31/519; A61P 35/00

(52) U.S. Cl. CPC A61K 31/4545; A61K 31/4196; A61P 35/00; A61K 31/565; A61K 31/519; A61K 31/566

(57) ABSTRACT

This invention relates to combination therapies comprising a cyclin dependent kinase (CDK) inhibitor, in particular a CDK4/6 inhibitor, and a proviral integration site for Moloney murine leukemia virus (PIM) inhibitor, and associated pharmaceutical compositions, methods of treatment, and uses.

### BACKGROUND OF THE INVENTION

### Field of the Invention

The present invention relates to combination therapies useful for the treatment of cancer. In particular, the invention relates to combination therapies comprising a cyclin dependent kinase (CDK) inhibitor and a proviral integration site for Moloney murine leukemia virus (PIM) inhibitor, optionally in further combination with an endocrine therapeutic agent. The invention also relates to associated methods of treatment, pharmaceutical compositions and pharmaceutical uses.

### Description of the Related Art

The PIM proteins are a family of oncogenic serine/threonine kinases including three isoforms, PIM1, PIM2 and PIM3, which are frequently amplified in cancer. PIM proteins are regulated primarily by transcription and stability through pathways that are controlled by Janus kinase/Signal transducer and activator of transcription (JAK/STAT) transcription factors. The PIM family proteins have been found to be overexpressed in hematological malignancies and solid tumors, and their roles in these tumors have been confirmed in mouse tumor models. The PIM family proteins have been implicated in the regulation of apoptosis, metabolism, cell cycle, and homing and migration, which has led to the postulation of these proteins as interesting targets for anticancer drug discovery. See Blanco-Aparicio & Carnero, Pim kinases in cancer: Diagnostic, prognostic and treatment opportunities, Biochem. Pharm. (2013) 85: 629-643. PIM1 is overexpressed in acute myeloid leukemia and PIM2 has been reported to play a role in multiple myeloma. Garcia, P. D. et al., Pan-PIM kinase inhibition provides a novel therapy for treating hematologic cancers, Clin. Cancer Res. (2014) 20: 1834-1845. PIM3 is overexpressed in certain cancers, including pancreatic cancer. Mukaida, N. et al., Roles of Pim-3, a novel survival kinase, in tumorigenesis, Cancer Sci. (2011) 102: 1437-1442.

The development of PIM inhibitors has been described in the literature. See, e.g., Narlik-Grassow, M. et al., The PIM family of serine/threonine kinases in cancer, Med. Res. Rev. (2014), 34: 136-159; Morwick, T. Pim kinase inhibitors: a survey of the patent literature, Expert Opin. Ther. Pat. (2010) 20: 193-212; Drygin, D. et al., Potential use of selective and non-selective Pim kinase inhibitors for cancer therapy, J. Med. Chem. (2012) 55: 8199-8208; and Arunesh, G. M. et al., Small molecule inhibitors of PIM1 kinase: July 2009 to February 2013 patent update, Expert. Opin. Ther. Pat. (2014) 24: 5-17.

Cyclin-dependent kinases (CDKs) and related serine/threonine protein kinases are important cellular enzymes that perform essential functions in regulating cell division and proliferation. CDKs 1-4, 6, 10, 11 have been reported to play a direct role in cell cycle progression, while CDKs 3, 5 and 7-9 may play an indirect role (e.g., through activation of other CDKs, regulation of transcription or neuronal functions). The CDK catalytic units are activated by binding to regulatory subunits, known as cyclins, followed by phosphorylation. Cyclins can be divided into four general classes (G1, G1/S, S and M cyclins) whose expression levels vary at different points in the cell cycle. Cyclin B/CDK1, cyclin A/CDK2, cyclin E/CDK2, cyclin D/CDK4, cyclin D/CDK6, and likely other heterodynes are important regulators of cell cycle progression.

The development of CDK inhibitors has been reviewed in the literature. For example, see Sanchez-Martinez et al., Cyclin dependent kinase (CDK) inhibitors as anticancer drugs, Bioorg. Med. Chem. Lett. (2015) 25: 3420-3435 (and references cited therein).

The use of cyclin dependent kinase 4/6 (CDK4/6) inhibitors in combination with endocrine therapy has demonstrated significant efficacy in the treatment of hormone receptor (HR)-positive, human epidermal growth factor 2 (HER2)-negative advanced or metastatic breast cancers, and CDK4/6 inhibitors, including palbociclib, ribociclib and abemaciclib, have been approved in combination with aromatase inhibitors in a first-line setting and fulvestrant in a second-line setting. Clinical investigation of CDK4/6 inhibitors in earlier stages of breast cancer and in other tumor types is ongoing.

Palbociclib and pharmaceutically acceptable salts and formulations thereof are disclosed in International Publication No. WO 2003/062236 and U.S. Pat. Nos. 6,936,612, RE47,739 and 7,456,168; International Publication No. WO 2005/005426 and U.S. Pat. Nos. 7,345,171 and 7,863,278; International Publication No. WO 2008/032157 and U.S. Pat. No. 7,781,583; International Publication No. WO 2014/128588; and International Publication No. WO 2016/193860. The contents of each of the foregoing references are incorporated herein by reference in their entirety.

Nevertheless, there remains a need for improved therapies for the treatment of cancer. The combinations and methods of the present invention are believed to have one or more advantages, such as greater efficacy in the treatment of certain cancers or subpopulations characterized by amplification, activation and/or overexpression of PIM kinases, as further described herein. These and other advantages of the present invention are apparent from the description below.

### BRIEF SUMMARY OF THE INVENTION

This invention relates to therapeutic methods, combinations and compositions for use in the treatment of abnormal cell growth, particularly cancer.

In one aspect, the invention provides a method of treating cancer in a subject in need thereof comprising administering to the subject an effective amount of a cyclin dependent kinase (CDK) inhibitor and an effective amount of a proviral integration site for Moloney murine leukemia virus (PIM) inhibitor. In some embodiments, the method further comprises administering to the subject an effective amount of an additional therapeutic agent.

In another aspect, the invention provides a method of treating cancer in a subject in need thereof comprising administering to the subject a cyclin dependent kinase (CDK) inhibitor, a proviral integration site for Moloney murine leukemia virus (PIM) inhibitor, and an additional therapeutic agent, wherein the amounts of the CDK inhibitor, the PIM inhibitor, and the additional therapeutic agent are together effective to treat the cancer.

In some embodiments of the aspects herein, the cancer is associated with PIM amplification, PIM activation and/or PIM overexpression. In some embodiments of the aspects herein, the CDK inhibitor is a CDK4/6 inhibitor. In some embodiments of the aspects herein, the PIM inhibitor is a PIM2 inhibitor. In frequent embodiments of the aspects herein, the CDK inhibitor is a CDK4/6 inhibitor and the PIM inhibitor is a PIM2 inhibitor. In some such embodiments, the cancer is associated with PIM2 amplification, PIM2 activation and/or PIM2 overexpression.

In preferred embodiments of each of the aspects and embodiments described herein, the CDK4/6 inhibitor is palbociclib, or a pharmaceutically acceptable salt thereof.

In another aspect, the invention provides a method of treating hormone receptor-positive (HR+), human epidermal growth factor receptor 2-negative (HER2−) breast cancer in a subject in need thereof, comprising administering to the subject a cyclin dependent kinase 4/6 (CDK4/6) inhibitor, a proviral integration site for Moloney murine leukemia virus 2 (PIM2) inhibitor, and an endocrine therapeutic agent, wherein the amounts of the CDK4/6 inhibitor, the PIM2 inhibitor, and the endocrine therapeutic agent are together effective to treat the cancer.

In some embodiments, the breast cancer is advanced or metastatic breast cancer. In some embodiments, the breast cancer is associated with PIM2 amplification, PIM2 activation and/or PIM2 overexpression.

In some embodiments of each of the aspects herein, the endocrine therapeutic agent is selected from the group consisting of an aromatase inhibitor, a selective estrogen receptor degrader (SERD), and a selective estrogen receptor modulator (SERM). In some embodiments, the endocrine therapeutic agent is selected from the group consisting of letrozole, anastrozole, exemestane and fulvestrant.

In another aspect, the invention provides a combination comprising a CDK inhibitor and a PIM inhibitor, wherein the combination is useful for the treatment of cancer in a subject in need thereof. In some embodiments, the cancer is associated with PIM amplification, PIM activation and/or PIM overexpression. In some such embodiments, the CDK inhibitor is a CDK4/6 inhibitor and the PIM inhibitor is a PIM2 inhibitor. In some such embodiments, the cancer is associated with PIM2 amplification, PIM2 activation and/or PIM2 overexpression. In some embodiments, the combination further comprises an endocrine therapeutic agent.

In some embodiments, the combination of the invention is useful for the treatment of HR+, HER2− breast cancer in a subject in need thereof. In some such embodiments, the breast cancer is advanced or metastatic breast cancer. In some embodiments, the cancer is associated with PIM amplification, PIM activation and/or PIM overexpression. In some such embodiments, the cancer is associated with PIM2 amplification, PIM2 activation and/or PIM2 overexpression.

In another aspect, the invention provides use of a combination comprising a CDK inhibitor and a PIM inhibitor for the treatment of cancer in a subject in need thereof. In some embodiments, the cancer is associated with PIM amplification, PIM activation and/or PIM overexpression. In some embodiments, the CDK inhibitor is a CDK4/6 inhibitor and the PIM inhibitor is a PIM2 inhibitor. In some such embodiments, the cancer is associated with PIM2 amplification, PIM2 activation and/or PIM2 overexpression. In some such embodiments, the combination further comprises use of an endocrine therapeutic agent.

In a further aspect, the invention provides a pharmaceutical composition comprising a CDK inhibitor, a PIM inhibitor, and a pharmaceutically acceptable carrier or excipient. In some such embodiments, the CDK inhibitor is a CDK4/6 inhibitor and the PIM inhibitor is a PIM2 inhibitor. In some such embodiments, the pharmaceutical composition further comprises an endocrine therapeutic agent.

In some embodiments of each of the aspects described herein, the CDK inhibitor is an inhibitor of cyclin dependent kinase 4 (CDK4) and/or cyclin dependent kinase 6 (CDK6). In particular embodiments, the CDK inhibitor is a CDK4/6 inhibitor (i.e., an inhibitor of both CDK4 and CDK6).

In preferred embodiments, the CDK4/6 inhibitor is palbociclib or a pharmaceutically acceptable salt thereof.

In other embodiments of each of the aspects described herein, the CDK4/6 inhibitor is ribociclib or abemaciclib, or a pharmaceutically acceptable salt thereof. In some embodiments, the CDK4/6 inhibitor is ribociclib or a pharmaceutically acceptable salt thereof. In some embodiments, the CDK4/6 inhibitor is abemaciclib or a pharmaceutically acceptable salt thereof.

In some embodiments, the PIM inhibitor is a PIM2 inhibitor. In some such embodiments, the PIM2 inhibitor further inhibits PIM1 and/or PIM3. In other embodiments, the PIM inhibitor is a pan-PIM inhibitor. In still other embodiments, the PIM inhibitor is a PIM1 inhibitor and/or a PIM3 inhibitor.

Each of the aspects and embodiments of the present invention described below may be combined with one or more other embodiments of the present invention described herein which is not inconsistent with the embodiment(s) with which it is combined.

### BRIEF DESCRIPTION OF THE DRAWINGS

FIG. 1A shows progression-free survival (PFS) by expression levels of PIM2 (higher or lower based on dichotomization by median) in the Study 1 cohort.

FIG. 1B shows PIM2 mRNA expression in two treatment arms of the Study 1 cohort.

FIG. 2A shows PFS by expression levels of PIM2 (higher or lower based on dichotomization by median) in the Study 2 cohort.

FIG. 2B shows PFS by expression levels of PIM2 in the Study 2 cohort, after adjusting for known clinicopathologic factors.

### DETAILED DESCRIPTION OF THE INVENTION

The present invention may be understood more readily by reference to the following detailed description of the preferred embodiments of the invention and the Examples included herein. It is to be understood that the terminology used herein is for the purpose of describing specific embodiments only and is not intended to be limiting. It is further to be understood that unless specifically defined herein, the terminology used herein is to be given its traditional meaning as known in the relevant art.

As used herein, the singular form “a”, “an”, and “the” include plural references unless indicated otherwise. For example, “a” substituent includes one or more substituents.

The invention described herein may be suitably practiced in the absence of any element(s) not specifically disclosed herein. Thus, in each instance herein where the term “comprising” is used in relation to any aspect or embodiment, the term may be replaced by “consisting essentially of” or “consisting of”.

The term “about” which used to modify a numerically defined parameter means that the parameter may vary by as much as 10% above or below the stated numerical value for that parameter. For example, a dose of about 5 mg/kg should be understood to mean that the dose may vary between 4.5 mg/kg and 5.5 mg/kg, unless otherwise specified.

The term “administration” and “treatment” as it applies to an animal, human, experimental subject, cell, tissue, organ or biological fluid, refers to contact of an exogenous pharmaceutical, therapeutic or diagnostic agent, or composition, to the animal, human, experimental subject, cell, tissue, organ or biological fluid. Treatment of a cell encompasses contact of a reagent to the cell, as well as contact of a reagent to a fluid, where the fluid is in contact with the cell. “Administration” and “treatment” also means in vitro and ex vivo treatment, e.g., of a cell, by a reagent, diagnostic, binding compound, or by another cell.

The terms “abnormal cell growth” and “hyperproliferative disorder” are used interchangeably in this application.

“Abnormal cell growth”, as used herein, unless otherwise indicated, refers to cell growth that is independent of normal regulatory mechanisms (e.g., loss of contact inhibition). Abnormal cell growth may be benign (not cancerous), or malignant (cancerous).

The term “cancer”, “cancerous”, “malignant” refer to or describe the physiological condition in mammals that is typically characterized by unregulated cell growth. As used herein “cancer” refers to any malignant and/or invasive growth or tumor caused by abnormal cell growth. As used herein “cancer” refers to solid tumors named for the type of cells that form them, cancer of blood, bone marrow, or the lymphatic system. Examples of solid tumors include but not limited to sarcomas and carcinomas. Examples of cancers of the blood include but not limited to leukemias, lymphomas and myeloma. The term “cancer” includes but is not limited to a primary cancer that originates at a specific site in the body, a metastatic cancer that has spread from the place in which it started to other parts of the body, a recurrence from the original primary cancer after remission, and a second primary cancer that is a new primary cancer in a person with a history of previous cancer of a different type from latter one. A subject may be identified as having de novo metastatic disease or after progression from an earlier-identified cancer.

The term “patient” or “subject” refer to any single subject for which therapy is desired or that is participating in a clinical trial, epidemiological study or used as a control, including humans and mammalian veterinary patients such as cattle, horses, dogs and cats. In preferred embodiments, the subject is a human.

In some embodiments, the subject is a postmenopausal woman or a man. In other embodiments, the subject is a pre- or perimenopausal woman treated with a luteinizing hormone releasing hormone (LNRH) agonist, such as goserelin, so that their ovarian function is suppressed. Subjects may be treatment naïve (i.e., the subject has not received prior treatment for advanced disease if metastatic, or for early disease if diagnosed with early breast cancer) or may have received one or more prior lines of treatment (i.e., in second or later line settings), such as one or more endocrine therapeutic agents or chemotherapeutic agents. In some embodiments, the subject is treated with an aromatase inhibitor as their initial endocrine therapeutic agent (i.e., as initial endocrine based therapy). In other embodiments, the subject has disease progression on or after treatment with an endocrine therapeutic agent in an adjuvant or metastatic setting.

The term “treat” or “treating” a cancer as used herein means to administer a combination therapy according to the present invention to a subject having cancer, or diagnosed with cancer, to achieve at least one positive therapeutic effect, such as, for example, reduced number of cancer cells, reduced tumor size, reduced rate of cancer cell infiltration into peripheral organs, or reduced rate of tumor metastases or tumor growth, reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above. The term “treating” also includes adjuvant and neo-adjuvant treatment of a subject.

For the purposes of this invention, beneficial or desired clinical results include, but are not limited to, one or more of the following: reducing the proliferation of (or destroying) neoplastic or cancerous cell; inhibiting metastasis or neoplastic cells; shrinking or decreasing the size of a tumor; remission of the cancer; decreasing symptoms resulting from the cancer; increasing the quality of life of those suffering from the cancer; decreasing the dose of other medications required to treat the cancer; delaying the progression of the cancer; curing the cancer; overcoming one or more resistance mechanisms of the cancer; and/or prolonging survival of patients the cancer. Positive therapeutic effects in cancer can be measured in a number of ways (see, for example, W. A. Weber, Assessing tumor response to therapy, J. Nucl. Med. 50 Suppl. 1:1S-10S (2009). For example, with respect to tumor growth inhibition (T/C), according to the National Cancer Institute (NCI) standards, a T/C less than or equal to 42% is the minimum level of anti-tumor activity. A T/C<10% is considered a high anti-tumor activity level, with T/C (%)=median tumor volume of the treated/median tumor volume of the control×100.

In some embodiments, the treatment achieved by a combination of the invention is defined by reference to any of the following: partial response (PR), complete response (CR), overall response (OR), progression free survival (PFS), disease free survival (DFS) and overall survival (OS). PFS, also referred to as “Time to Tumor Progression” indicates the length of time during and after treatment that the cancer does not grow and includes the amount of time patients have experienced a CR or PR, as well as the amount of time patients have experienced stable disease (SD). DFS refers to the length of time during and after treatment that the patient remains free of disease. OS refers to a prolongation in life expectancy as compared to naïve or untreated subjects or patients. In some embodiments, response to a combination of the invention is any of PR, CR, PFS, DFS, OR or OS that is assessed using Response Evaluation Criteria in Solid Tumors (RECIST) 1.1 response criteria.

The treatment regimen for a combination of the invention that is effective to treat a cancer patient may vary according to factors such as the disease state, age, and weight of the patient, and the ability of the therapy to elicit an anti-cancer response in the subject.

While an embodiment of any of the aspects of the invention may not be effective in achieving a positive therapeutic effect in every subject, it should do so in a statistically significant number of subjects as determined by any statistical test known in the art such as the Student's t-test, the chi2-test the U-test according to Mann and Whitney, the Kruskal-Wallis test (H-test), Jonckheere-Terpstrat-testy and the Wilcon on-test.

The terms “treatment regimen”, “dosing protocol” and “dosing regimen” are used interchangeably to refer to the dose and timing of administration of each therapeutic agent in a combination of the invention.

“Ameliorating” means a lessening or improvement of one or more symptoms upon treatment with a combination described herein, as compared to not administering the combination. “Ameliorating” also includes shortening or reduction in duration of a symptom.

As used herein, an “effective dosage” or “effective amount” of drug, compound or pharmaceutical composition is an amount sufficient to affect any one or more beneficial or desired, including biochemical, histological and/or behavioral symptoms, of the disease, its complications and intermediate pathological phenotypes presenting during development of the disease. For therapeutic use, a “therapeutically effective amount” refers to that amount of a compound being administered which will relieve to some extent one or more of the symptoms of the disorder being treated. In reference to the treatment of cancer, a therapeutically effective amount refers to that amount which has the effect of (1) reducing the size of the tumor, (2) inhibiting (that is, slowing to some extent, preferably stopping) tumor metastasis, (3) inhibiting to some extent (that is, slowing to some extent, preferably stopping) tumor growth or tumor invasiveness, (4) relieving to some extent (or, preferably, eliminating) one or more signs or symptoms associated with the cancer, (5) decreasing the dose of other medications required to treat the disease, and/or (6) enhancing the effect of another medication, and/or (7) delaying the progression of the disease in a patient.

An effective dosage can be administered in one or more administrations. For the purposes of this invention, an effective dosage of drug, compound, or pharmaceutical composition is an amount sufficient to accomplish prophylactic or therapeutic treatment either directly or indirectly. As is understood in the clinical context, an effective dosage of drug, compound or pharmaceutical composition may or may not be achieved in conjunction with another drug, compound or pharmaceutical composition.

A cancer or biological sample which displays PIM amplification, PIM activation and/or PIM overexpression is one which, in a diagnostic test, expresses (including overexpresses) a PIM protein, has an amplified PIM gene, and/or otherwise demonstrates activation of PIM, which can be determined directly or indirectly. Such cancers typically have significantly higher levels of a PIM protein or gene compared to noncancerous cells of the same tissue type. For example, overexpression may be caused by gene amplification or by increased transcription or translation.

PIM overexpression or amplification may be determined in a diagnostic or prognostic assay by evaluating increased levels of the PIM protein, e.g. via an immunohistochemistry (IHC) assay, Western blot analysis, immunoprecipitation, molecular binding assays, ELISA, ELIFA, fluorescence activated cell sorting (“FACS”), proteomics, quantitative blood based assays (e.g., Serum ELISA), or biochemical enzymatic activity assays. Alternatively, one may measure levels of PIM-encoding nucleic acid, e.g. via in situ hybridization (ISH) methods (including FISH), Southern blotting, Northern analysis, or polymerase chain reaction (PCR) techniques, including quantitative real time PCR (“qRT-PCR”) and other amplification type detection methods, such as, for example, branched DNA, SISBA, TMA and the like, or RNA-Seq, MassARRAY, microarray analysis, gene expression profiling, and/or serial analysis of gene expression (“SAGE”). A wide variety of assays can be performed by protein, gene, and/or tissue array analysis. In addition, in vivo assays are available to the skilled practitioner. For example, one may expose cells from a biological sample from a subject to an antibody, which is optionally labeled with a detectable label (e.g. a radioactive isotope) and determining the binding of the antibody to cells in sample, e.g. by external scanning for radioactivity or by analyzing a biopsy from a subject previously exposed to the antibody.

“Tumor” as it applies to a subject diagnosed with, or suspected of having, a cancer refers to a malignant or potentially malignant neoplasm or tissue mass of any size and includes primary tumors and secondary neoplasms. A solid tumor is an abnormal growth or mass of tissue that usually does not contain cysts or liquid areas. Examples of solid tumors are sarcomas, carcinomas, and lymphomas. Leukaemia's (cancers of the blood) generally do not form solid tumors (National Cancer Institute, Dictionary of Cancer Terms).

“Tumor burden” or “tumor load”, refers to the total amount of tumorous material distributed throughout the body. Tumor burden refers to the total number of cancer cells or the total size of tumor(s), throughout the body, including lymph nodes and bone marrow. Tumor burden can be determined by a variety of methods known in the art, such as, e.g., using calipers, or while in the body using imaging techniques, e.g., ultrasound, bone scan, computed tomography (CT), or magnetic resonance imaging (MRI) scans.

The term “tumor size” refers to the total size of the tumor which can be measured as the length and width of a tumor. Tumor size may be determined by a variety of methods known in the art, such as, e.g., by measuring the dimensions of tumor(s) upon removal from the subject, e.g., using calipers, or while in the body using imaging techniques, e.g., bone scan, ultrasound, CR or MRI scans.

The term “additive” is used to mean that the result of the combination of two compounds, components or targeted agents is no greater than the sum of each compound, component or targeted agent individually.

The term “synergy” or “synergistic” are used to mean that the result of the combination of two compounds, components or targeted agents is greater than the sum of each compound, component or targeted agent individually. This improvement in the disease, condition or disorder being treated is a “synergistic” effect. A “synergistic amount” is an amount of the combination of the two compounds, components or targeted agents that results in a synergistic effect, as “synergistic” is defined herein.

Determining a synergistic interaction between one or two components, the optimum range for the effect and absolute dose ranges of each component for the effect may be definitively measured by administration of the components over different dose ranges, and/or dose ratios to patients in need of treatment. However, the observation of synergy in in vitro models or in vivo models can be predictive of the effect in humans and other species and in vitro models or in vivo models exist, as described herein, to measure a synergistic effect. The results of such studies can also be used to predict effective dose and plasma concentration ratio ranges and the absolute doses and plasma concentrations required in humans and other species such as by the application of pharmacokinetic and/or pharmacodynamics methods.

CDK inhibitors useful in the invention include CDK4 inhibitors, CDK6 inhibitors, and CDK4/6 inhibitors. Such compounds may be pan-CDK inhibitors, which inhibit multiple CDKs, or may selectively inhibit CDK4 and/or CDK6. CDK inhibitors may have activity against targets in addition to CDKs. In preferred embodiments, the CDK4/6 inhibitor is palbociclib, or a pharmaceutically acceptable salt thereof. PIM inhibitors useful in the invention include PIM1, PIM2 and PIM3 inhibitors. Such compounds may be selective PIM inhibitors, which preferentially inhibit a single PIM, or pan-PIM inhibitors, which inhibit multiple PIM targets. In some embodiments, the PIM inhibitor is a PIM2 inhibitor. In some embodiments, the PIM2 inhibitor further inhibits PIM1 and/or PIM3. In some embodiments, the PIM inhibitor is a PIM1 inhibitor. In some embodiments, the PIM inhibitor is a PIM3. In other embodiments, the PIM inhibitor is a pan-PIM inhibitor.

Examples of pan-PIM inhibitors include: AZD1208 (Dakin, L. A. et al., Discovery of novel benzylidene-1,3-thiazolidine-2,4-diones as potent and selective inhibitors of the PIM-1, PIM-2, and PIM-3 protein kinases. Bioorg. Med. Chem. Lett. (2012) 22: 4599-4604); PIM447 (or LGH477) (Burger, M. T. et al., Identification of N-(4-((1R,3S,5S)-3-Amino-5-methylcyclohexyl)pyridine-3-yl)-6-(2,6-difluorophenyl)-5-fluoropicolinamide (PIM447), a potent and selective proviral insertion site of Moloney murine leukemia (PIM) 1, 2, and 3 kinase inhibitor in clinical trials for hematological malignancies. J. Med. Chem. (2015) 58: 8373-8386; TP-3654 (Foulks, J. M., A Small-Molecule Inhibitor of PIM Kinases as a Potential Treatment for Urothelial Carcinomas, Neoplasia (2014) 16(5): 403-412; INCB053914 (Koblish, H. et al., Preclinical characterization of INCB053914, a novel pan-PIM kinase inhibitor, alone and in combination with anticancer agents, in models of hematologic malignancies, PLoS One. (2018) 21; 13(6):e0199108; SGI-1776 (Chen L. S., Pim kinase inhibitor, SGI-1776, induces apoptosis in chronic lymphocytic leukemia cells, Blood (2009) 114:4150-4157; and LGB321 (Garcia et al., (2014). See also Warfel, N. A. and Kraft, A. S., PIM kinase (and Akt) biology and signaling in tumors, Pharmacology & Therapeutics, (2015) 151: 41-49 and references cited therein. Other classes of PIM inhibitors are described in WO 2012/154274.

Unless indicated otherwise, all references herein to CDK inhibitors and PIM inhibitors include references to pharmaceutically acceptable salts, solvates, hydrates and complexes thereof, and to solvates, hydrates and complexes of salts thereof, and include amorphous and polymorphic forms, stereoisomers, and isotopically labeled versions thereof.

As used herein, the term “pharmaceutically acceptable salt” refers to those salts which retain the biological effectiveness and properties of the parent compound. The phrase “pharmaceutically acceptable salt(s)”, as used herein, unless otherwise indicated, includes salts of acidic or basic groups which may be present in the compounds of the formulae disclosed herein.

For example, the compounds of the invention that are basic in nature may be capable of forming a wide variety of salts with various inorganic and organic acids. The acids that may be used to prepare pharmaceutically acceptable acid addition salts of such basic compounds of those that form non-toxic acid addition salts, i.e., salts containing pharmacologically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate [i.e., 1,1′-methylene-bis-(2-hydroxy-3-naphthoate)] salts. Examples of salts include, but are not limited to, acetate, acrylate, benzenesulfonate, benzoate (such as chlorobenzoate, methylbenzoate, dinitrobenzoate, hydroxybenzoate, and methoxybenzoate), bicarbonate, bisulfate, bisulfite, bitartrate, borate, bromide, butyne-1,4-dioate, calcium edetate, camsylate, carbonate, chloride, caproate, caprylate, clavulanate, citrate, decanoate, dihydrochloride, dihydrogenphosphate, edetate, edislyate, estolate, esylate, ethylsuccinate, formate, fumarate, gluceptate, gluconate, glutamate, glycollate, glycollylarsanilate, heptanoate, hexyne-1,6-dioate, hexylresorcinate, hydrabamine, hydrobromide, hydrochloride, γ-hydroxybutyrate, iodide, isobutyrate, isothionate, lactate, lactobionate, laurate, malate, maleate, malonate, mandelate, mesylate, metaphosphate, methane-sulfonate, methylsulfate, monohydrogenphosphate, mucate, napsylate, naphthalene-1-sulfonate, naphthalene-2-sulfonate, nitrate, oleate, oxalate, pamoate (embonate), palmitate, pantothenate, phenylacetates, phenylbutyrate, phenylpropionate, phthalate, phosphate/diphosphate, polygalacturonate, propanesulfonate, propionate, propiolate, pyrophosphate, pyrosulfate, salicylate, stearate, subacetate, suberate, succinate, sulfate, sulfonate, sulfite, tannate, tartrate, teoclate, tosylate, triethiodode, and valerate salts.

Alternatively, the compounds useful that are acidic in nature may be capable of forming base salts with various pharmacologically acceptable cations. Examples of such salts include the alkali metal or alkaline-earth metal salts and particularly, the sodium and potassium salts. These salts may be prepared by conventional techniques. The chemical bases which may be used as reagents to prepare the pharmaceutically acceptable base salts of this invention include those which form non-toxic base salts with the acidic compounds herein. The chemical bases that may be used as reagents to prepare pharmaceutically acceptable base salts of the compounds of the invention that are acidic in nature are those that form non-toxic base salts with such compounds. Such non-toxic base salts include, but are not limited to, those derived from such pharmacologically acceptable cations such as alkali metal cations (e.g., potassium and sodium) and alkaline earth metal cations (e.g., calcium and magnesium), ammonium or water-soluble amine addition salts such as N-methylglucamine-(meglumine), and the lower alkanolammonium and other base salts of pharmaceutically acceptable organic amines. Hemisalts of acids and bases may also be formed, for example, hemisulphate and hemicalcium salts. For a review on suitable salts, see Handbook of Pharmaceutical Salts: Properties, Selection, and Use by Stahl and Wermuth (Wiley-VCH, 2002). Methods for making pharmaceutically acceptable salts are known to those of skill in the art.

### Gene Expression Analysis

Tumor samples were collected from subjects having ER+/HR+, HER2− breast cancer who were participants in placebo-controlled clinical studies of palbociclib in combination with letrozole (Study 1) or fulvestrant (Study 2). The studies were conducted in different treatment populations. Study 1 enrolled post-menopausal women with ER+/HER2− advanced breast cancer who had not received prior systematic therapy for their advanced disease. Subjects were treated with palbociclib (125 mg QD, 3/1 schedule) plus letrozole (2.5 mg QD) in the treatment arm, or placebo (3/1 schedule QD) plus letrozole (2.5 mg QD) in the control arm.

Study 2 was conducted in HR+/HER2− advanced or metastatic breast cancer (MBC) patients who had progressed on prior endocrine therapy. Pre- and peri-menopausal subjects were given an LHRH agonist. Subjects were treated with palbociclib (125 mg QD, 3/1 schedule) plus fulvestrant (500 mg IM on Days 1 and 15 of Cycle 1, and then Day 1 of each subsequent 28-day cycle) or placebo (3/1 schedule QD) plus fulvestrant on the same schedule. In both studies, the primary outcome was PFS as assessed by the investigator.

Tumor samples were analyzed to identify potential predictive biomarkers for palbociclib plus letrozole treatment. Using the EdgeSeq Oncology BM Panel, mRNA expression was assessed for 2534 cancer-related genes. Cox regression analysis was performed to investigate potential interaction between biomarker levels, as either a continuous variable or dichotomized by median level, and treatment effect in terms of progression-free survival (PFS). Targeted capture sequencing was used to quantitate RNA expression levels of gene targets.

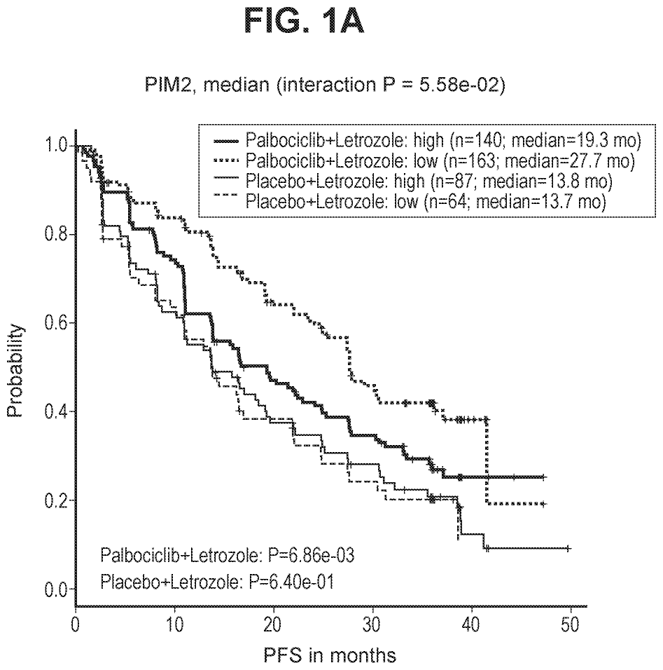

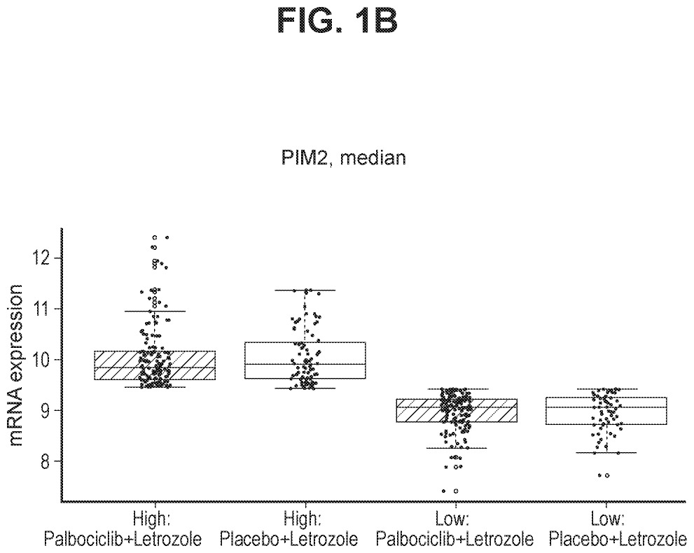

The gene expression analysis was performed blinded to the clinical information. When subsequently correlated to clinical outcomes, the analysis resulted in the identification of PIM2 expression as being associated with resistance to the combination therapy. Subjects having higher levels of PIM2 expression demonstrated a shorter PFS in the palbociclib plus letrozole treatment arm than subjects identified as having a lower level of PIM2 expression. FIG. 1A shows PFS by expression levels of PIM2 (higher or lower based on dichotomization by median) in the Study 1 cohort. FIG. 1B shows PIM2 expression comparison in two treatment arms of the Study 1 cohort. The level of PIM2 expression did not appear to have a significant impact on PFS for subjects in the control arm, who were treated with letrozole alone. These subjects had a similar PFS regardless of whether they were in the higher or lower PIM2 expression groups, as shown in FIG. 1A. PIM2 was also observed to be more highly expressed in palbociclib resistant HER2E subtype in Study 1.

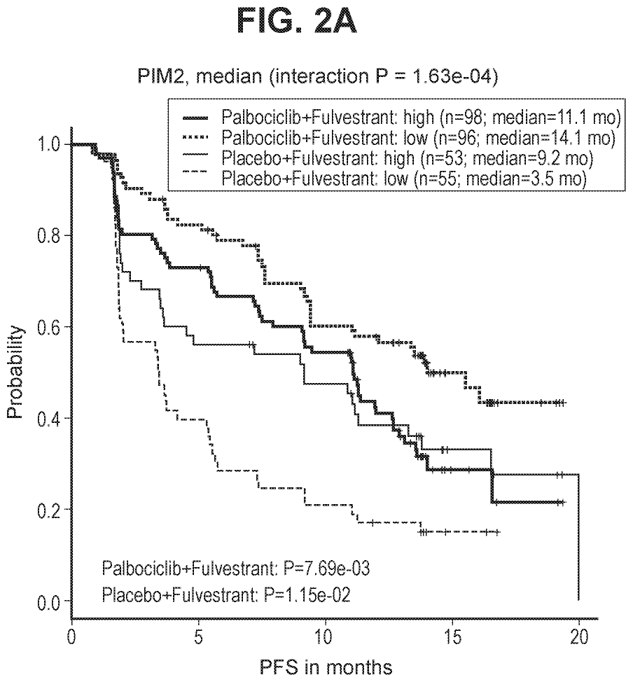

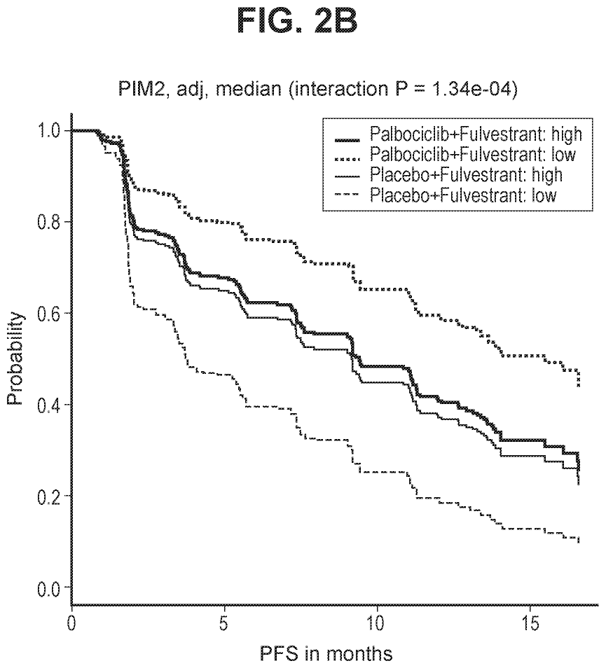

A similar analysis was conducted on samples collected for subjects in Study 2, validating PIM2 as a predictive resistance biomarker in the subsequent independent Study 2 cohort. FIG. 2A shows PFS by expression levels of PIM2 (higher or lower based on dichotomization by median) in the Study 2 cohort. FIG. 2B shows PFS by expression levels of PIM2 in the Study 2 cohort after adjusting for known clinicopathologic factors (visceral metastases, site of tissue collection, baseline ECOG performance status, recurrence type, prior chemotherapy, prior tamoxifen, and prior AI).

The magnitude of relative palbociclib benefit was found to be smaller in patients with higher PIM2 mRNA expression in both the Study 2 (FIG. 2A; continuous interaction P=0.0150) and Study 1 (FIG. 1A; continuous interaction P=0.0785) cohorts, despite similar levels of expression between the two arms (FIG. 1B) and accounting for baseline clinicopathological characteristics (FIG. 2B). In contrast to the results in Study 1, subjects in the control arm of Study 2 who received fulvestrant alone showed a significant difference in PFS depending on whether they were in the high or low PIM2 expression subgroups. Subjects having low PIM2 expression had shorter PFS when treated with fulvestrant alone than those having higher PIM2 expression receiving fulvestrant alone (FIG. 2A). The reason for the difference is not fully understood, but subjects in Study 2 had progressed on prior endocrine therapy.

### Therapeutic Methods, Uses, Combinations and Compositions

In one aspect, the invention provides a method of treating cancer in a subject in need thereof comprising administering to the subject an effective amount of a cyclin dependent kinase (CDK) inhibitor and an effective amount of a proviral integration site for Moloney murine leukemia virus (PIM) inhibitor. In some embodiments, the method further comprises administering to the subject an effective amount of an additional therapeutic agent.

In some embodiments of the foregoing methods, the cancer is associated with PIM amplification, PIM activation and/or PIM overexpression. In some such embodiments, the subject is identified as having PIM amplification, PIM activation and/or PIM overexpression.

In another aspect, the invention provides a combination comprising a CDK inhibitor and a PIM inhibitor. In another aspect, the invention provides a combination comprising a CDK inhibitor and a PIM inhibitor for use in the treatment of cancer. In another aspect, the invention provides a combination comprising a CDK inhibitor and a PIM inhibitor, wherein the combination is useful for the treatment of cancer. In another aspect, the invention provides a combination comprising a CDK inhibitor and a PIM inhibitor for use as a medicament. In some embodiments, the medicament is for the treatment of cancer.

In some embodiments of each of the foregoing aspects, the combination further comprises an additional therapeutic agent.

In another aspect, the invention provides use of a combination comprising a CDK inhibitor and a PIM inhibitor for the treatment of cancer. In another aspect, the invention provides use of a combination comprising a CDK inhibitor and a PIM inhibitor for the manufacture of a medicament. In some embodiments, the medicament is for the treatment of cancer.

In some embodiments of the foregoing, use of the combination further comprises use of an additional therapeutic agent.

In some embodiments of the foregoing combinations and uses, the cancer is associated with PIM amplification, PIM activation and/or PIM overexpression.

In some embodiments of the foregoing aspects, the CDK inhibitor is an inhibitor of cyclin dependent kinase 4 (CDK4) and/or cyclin dependent kinase 6 (CDK6). In other embodiments of each of the foregoing aspects, the CDK inhibitor is a CDK4 inhibitor, a CDK6 inhibitor or a CDK4/6 inhibitor. In specific embodiments, the CDK inhibitor is a CDK4/6 inhibitor. In a preferred embodiment, the CDK4/6 inhibitor is palbociclib or a pharmaceutically acceptable salt thereof.

In some embodiments of the foregoing aspects, the PIM inhibitor is an inhibitor of PIM1, PIM2 or PIM3. In some embodiments of the foregoing, the PIM inhibitor is a PIM2 inhibitor. In some such embodiments, the PIM2 inhibitor further inhibits PIM1 and/or PIM3. In other embodiments, the PIM inhibitor is a PIM1 inhibitor. In some embodiments, the PIM inhibitor is an inhibitor of PIM3. In some embodiments, the PIM inhibitor is a pan-PIM inhibitor.

In preferred embodiments of each of the foregoing methods, combinations and uses, the CDK inhibitor is a CDK4/6 inhibitor and the PIM inhibitor is a PIM2 inhibitor. In particularly preferred embodiments, the CDK4/6 inhibitor is palbociclib or a pharmaceutically acceptable salt thereof, and the PIM inhibitor is a PIM2 inhibitor. In some such embodiments, the cancer is associated with PIM2 amplification, PIM2 activation and/or PIM2 overexpression.

In another aspect, the invention provides a method of treating cancer in a subject in need thereof comprising administering to the subject an effective amount of a CDK 4/6 inhibitor and an effective amount of a PIM2 inhibitor. In some embodiments, the method further comprises administering to the subject an effective amount of an additional therapeutic agent.

In some embodiments of the foregoing methods, the cancer is associated with PIM2 amplification, PIM2 activation and/or PIM2 overexpression. In some such embodiments, the subject is identified as having PIM2 amplification, PIM2 activation and/or PIM2 overexpression.

In another aspect, the invention provides a combination comprising a CDK4/6 inhibitor and a PIM2 inhibitor. In another aspect, the invention provides a combination comprising a CDK4/6 inhibitor and a PIM2 inhibitor for use in the treatment of cancer. In another aspect, the invention provides a combination comprising a CDK4/6 inhibitor and a PIM2 inhibitor, wherein the combination is useful for the treatment of cancer in a subject in need thereof. In some embodiments of each of the foregoing aspects, the combination further comprises an additional therapeutic agent.

In another aspect, the invention provides use of a combination comprising a CDK4/6 inhibitor and a PIM2 inhibitor for the treatment of cancer. In some embodiments of the foregoing, use of the combination further comprises use of an additional therapeutic agent, in particular an endocrine therapeutic agent.

In some embodiments of the foregoing combinations and uses, the cancer is associated with PIM2 amplification, PIM2 activation and/or PIM2 overexpression.

In some embodiments of each of the foregoing combinations and uses, the combination is a synergistic combination.

In another aspect, the invention provides a pharmaceutical composition comprising a CDK inhibitor, a PIM inhibitor, and a pharmaceutically acceptable carrier or excipient. In some embodiments, the invention provides a pharmaceutical composition comprising a CDK4/6 inhibitor, a PIM2 inhibitor inhibitor, and a pharmaceutically acceptable carrier or excipient.

In another aspect, the invention provides a method of treating hormone receptor-positive (HR+), human epidermal growth factor receptor 2-negative (HER2-) breast cancer in a subject in need thereof, comprising administering to the subject a cyclin dependent kinase 4/6 (CDK4/6) inhibitor, a proviral integration site for Moloney murine leukemia virus 2 (PIM2) inhibitor, and optionally an endocrine therapeutic agent, wherein the amounts of the CDK4/6 inhibitor, the PIM2 inhibitor, and the optional endocrine therapeutic agent are together effective to treat cancer.

In another aspect, the invention provides a method of treating hormone receptor-positive (HR+), human epidermal growth factor receptor 2-negative (HER2−) breast cancer in a subject in need thereof, comprising administering to the subject a cyclin dependent kinase 4/6 (CDK4/6) inhibitor, a proviral integration site for Moloney murine leukemia virus 2 (PIM2) inhibitor, and an endocrine therapeutic agent, wherein the amounts of the CDK4/6 inhibitor, the PIM2 inhibitor, and the endocrine therapeutic agent are together effective to treat cancer.

In another aspect, the invention provides a method of treating HR+, HER2− breast cancer in a subject in need thereof, consisting essentially of administering to the subject a CDK4/6 inhibitor, a PIM2 inhibitor, and an endocrine therapeutic agent, wherein the amounts of the CDK4/6 inhibitor, the PIM2 inhibitor, and the endocrine therapeutic agent are together effective to treat the cancer.

In another aspect, the invention provides a method of treating HR+, HER2− breast cancer in a subject in need thereof, consisting of administering to the subject a CDK4/6 inhibitor, a PIM2 inhibitor, and an endocrine therapeutic agent, wherein the amounts of the CDK4/6 inhibitor, the PIM2 inhibitor, and the endocrine therapeutic agent are together effective to treat the cancer.

In some embodiments of each of the aspects herein, the breast cancer is advanced or metastatic breast cancer. In other embodiments of each of the aspects herein, the breast cancer is early breast cancer. In some embodiments of each of the aspects herein, the breast cancer is advanced or metastatic breast cancer, or early breast cancer. In some embodiments of each of the aspects herein, the breast cancer is associated with PIM2 amplification, PIM2 activation and/or PIM2 overexpression.

In some embodiments of each of the aspects herein, the PIM2 inhibitor further inhibits PIM1 and/or PIM3. In some such embodiments, the PIM2 inhibitor is a pan-PIM inhibitor (i.e., it inhibits PIM1, PIM2 and PIM3).

In some embodiments of each of the aspects herein, the endocrine therapeutic agent is selected from the group consisting of an aromatase inhibitor, a selective estrogen receptor degrader (SERD), and a selective estrogen receptor modulator (SERM). In some embodiments, the endocrine therapeutic agent is selected from the group consisting of letrozole, anastrozole, exemestane and fulvestrant.

In some embodiments of each of the aspects described herein, the cancer is breast cancer. In some such embodiments of each of the aspects described herein, the breast cancer is advanced or metastatic breast cancer. In other such embodiments, the breast cancer is early breast cancer.

In some embodiments of each of the aspects herein, the cancer is associated with PIM amplification, PIM activation and/or PIM overexpression. In some such embodiments of each of the aspects herein, the cancer is associated with PIM2 amplification, PIM2 activation and/or PIM2 overexpression.

In some embodiments of each of the aspects herein, the PIM inhibitor is a PIM2 inhibitor. In some such embodiments of each of the aspects herein, the PIM2 inhibitor further inhibits PIM1 and/or PIM3. In some such embodiments, the PIM2 inhibitor is a pan-PIM inhibitor.

In some embodiments of each of the aspects herein, the endocrine therapeutic agent is selected from the group consisting of an aromatase inhibitor, a selective estrogen receptor degrader (SERD), and a selective estrogen receptor modulator (SERM).

In some embodiments, the endocrine therapeutic agent is an aromatase inhibitor. In some embodiments, the aromatase inhibitor is selected from the group consisting of letrozole, anastrozole and exemestane. In some such embodiments, the aromatase inhibitor is letrozole. In some embodiments, the aromatase inhibitor is a nonsteroidal aromatase inhibitor (NSAI). In some such embodiments, the nonsteroidal aromatase inhibitor is letrozole or anastrozole. In some embodiments, the aromatase inhibitor is a steroidal aromatase inhibitor (SAI). In some such embodiments, the steroidal aromatase inhibitor is exemestane.

In other embodiments, the endocrine therapeutic agent is a SERD. In some embodiments, the SERD is selected from the group consisting of fulvestrant, brilanestrant and elacestrant. In some such embodiments, the SERD is fulvestrant. In some embodiments, the endocrine therapeutic agent is a SERM. In some such embodiments, the SERM is selected from the group consisting of tamoxifen, lasofoxifene, bazedoxifene and toremifene.

In still other embodiments of each of the aspects herein, the endocrine therapeutic agent is selected from the group consisting of letrozole, anastrozole, exemestane and fulvestrant. In still other embodiments the endocrine therapeutic agent is letrozole. In still other embodiments the endocrine therapeutic agent is fulvestrant. In preferred embodiments of each of the aspects herein, the CDK 4/6 inhibitor is palbociclib, or a pharmaceutically acceptable salt thereof.

In some embodiments, the CDK4/6 inhibitor and the PIM2 inhibitor are administered sequentially, simultaneously or concurrently.

In another aspect, the invention provides a combination comprising a CDK4/6 inhibitor and a PIM2 inhibitor. In some embodiments, the combination is useful for the treatment of HR+, HER2− breast cancer in a subject in need thereof. In some embodiments, the combination optionally further comprises an endocrine therapeutic agent. In some embodiments, the combination is a synergistic combination.

In another aspect, the invention provides a combination consisting essentially of a CDK4/6 inhibitor, a PIM2 inhibitor and optionally an endocrine therapeutic agent. In a further aspect, the invention provides a pharmaceutical composition comprising a CDK4/6 inhibitor, a PIM2 inhibitor, and a pharmaceutically acceptable carrier or excipient.

The methods, uses, combinations and compositions of the present inventions may be useful for treating cancer. Some embodiments provided herein result in one or more of the following effects: (1) inhibiting cancer cell proliferation; (2) inhibiting cancer cell invasiveness; (3) inducing apoptosis of cancer cells; (4) inhibiting cancer cell metastasis; (5) inhibiting angiogenesis; or (6) overcoming one or more resistance mechanisms relating to a cancer treatment.

In another aspect, the invention provides a method of treating cancer in a subject in need thereof comprising administering to the subject a combination therapy comprising a CDK inhibitor and a PIM inhibitor.

In another aspect, the invention provides a method of treating cancer in a subject in need thereof comprising administering to the subject a combination therapy comprising an effective amount of a CDK4/6 inhibitor and an effective amount of a PIM2 inhibitor.

In some embodiments of each of the methods described herein, the subject is identified as having a cancer associated with PIM amplification, PIM activation and/or PIM overexpression. In some such embodiments, the subject is identified as having a cancer associated with PIM2 amplification, PIM2 activation and/or PIM2 overexpression.

Some embodiments of the therapeutic methods described herein further comprise administering to the subject an amount of one or more additional therapeutic agents. In some such embodiments, the amounts of the CDK inhibitor, the PIM inhibitor and the additional therapeutic agent(s) are together effective to treat cancer. In some such embodiments, the one or more additional therapeutic agents are selected from the group consisting of endocrine therapy (e.g., an aromatase inhibitor, a SERD, a SERM, an anti-androgen agent), a PI3K inhibitor, an mTOR inhibitor, a PARP inhibitor, a TGF-β inhibitor or an anti-PD axis antagonist (e.g., a PD-1 or PD-L1 antagonist). In frequent embodiments, the one or more additional therapeutic agents is an endocrine therapy, in particular letrozole or fulvestrant. In some such embodiments, the methods and uses of the invention comprise administering to the subject an amount of one or more additional therapeutic agents, with the proviso that the list of one or more additional anti-cancer agents does not comprise a JAK inhibitor. In another aspect, the invention provides a composition for use in the treatment of cancer comprising a CDK inhibitor and a PIM inhibitor, for example a CDK4/6 inhibitor and a PIM2 inhibitor. In another aspect, the invention provides a composition for use in the treatment of cancer comprising a CDK inhibitor, a PIM inhibitor, and a pharmaceutically acceptable carrier or excipient. In particular embodiments, the pharmaceutical composition further comprises one or more additional therapeutic agents, including embodiments as further described herein.

In some embodiments of the aspects described herein, the cancer is associated with PIM amplification, PIM activation and/or PIM overexpression. In some embodiments, the cancer associated with PIM amplification, PIM activation and/or PIM overexpression is rated by gene expression of tumor tissues and/or by immunohistochemical scores corresponding to the number of copies of PIM molecules expressed per cell. In some such embodiments, the cancer associated with PIM2 amplification, PIM2 activation and/or PIM2 overexpression is rated by gene expression of tumor tissues and/or by immunohistochemical scores corresponding to the number of copies of PIM2 molecules expressed per cell.

In another aspect, the invention provides a method of selecting a subject having cancer for treatment with a combination of a CDK inhibitor and a PIM inhibitor, comprising: (a) detecting the presence of PIM amplification, PIM activation and/or PIM overexpression in a biological sample from the subject; and (b) selecting the subject for treatment with the combination of a CDK inhibitor and a PIM inhibitor. Particular embodiments further comprise (c) administering an effective amount of a CDK inhibitor and a PIM inhibitor to the subject.

In another aspect, the invention provides a method of selecting a subject having cancer for treatment with a combination of a CDK4/6 inhibitor and a PIM2 inhibitor, comprising: (a) detecting the presence of PIM2 amplification, PIM2 activation and/or PIM2 overexpression in a biological sample from the subject; and (b) selecting the subject for treatment with the combination of a CDK4/6 inhibitor and a PIM2 inhibitor. Particular embodiments further comprise (c) administering an effective amount of a CDK4/6 inhibitor and a PIM2 inhibitor to the subject.

In another aspect, the invention provides a method of selecting a subject having cancer for treatment with a combination of a CDK4/6 inhibitor, a PIM2 inhibitor, and optionally an endocrine therapeutic agent, comprising: (a) detecting the presence of PIM2 amplification, PIM2 activation and/or PIM2 overexpression in a biological sample from the subject; and (b) selecting the subject for treatment with the combination of a CDK4/6 inhibitor, a PIM2 inhibitor, and optionally an endocrine therapeutic agent. Particular embodiments further comprise (c) administering an effective amount of a CDK4/6 inhibitor, a PIM2 inhibitor, and optionally an endocrine therapeutic agent to the subject.

In another aspect, the invention provides a method of predicting whether a subject having cancer will be resistant to treatment with a CDK4/6 inhibitor, comprising comparing the level of PIM expression in a biological sample from the subject to the level of PIM expression in a control sample, wherein increased PIM expression in the subject sample relative to the control sample indicates the subject is likely to be resistant to treatment with the CDK4/6 inhibitor. In some embodiments of this aspect, the invention further provides administering an effective amount of a CDK4/6 inhibitor and a PIM inhibitor to the subject. In some embodiments of this aspect, the invention further provides treating the subject with a combination comprising a CDK4/6 inhibitor and a PIM inhibitor. Specific embodiments of this aspect relate to analysing the level of PIM2 expression and treatment with a PIM2 inhibitor.

In another aspect, the invention provides a method of predicting whether a subject having cancer will be resistant to treatment with a CDK4/6 inhibitor and an endocrine therapeutic agent, comprising comparing the level of PIM expression in a biological sample from the subject to the level of PIM expression from a control, wherein increased PIM expression in the subject sample relative to the control indicates the subject is likely to be resistant to treatment with the CDK4/6 inhibitor and the endocrine therapeutic agent. In some embodiments of this aspect, the invention further provides administering an effective amount of a CDK4/6 inhibitor, an endocrine therapeutic agent and a PIM inhibitor to the subject. In some embodiments, the control may be a normal control from an individual or population, or a non-tumor comparator from patient. Specific embodiments of this aspect relate to analysing the level of PIM2 expression and treatment with a PIM2 inhibitor.

In another aspect, the invention provides a method of treating cancer in a subject in need thereof, comprising: (a) detecting the presence of PIM amplification, PIM activation and/or PIM overexpression in a biological sample from the subject; (b) selecting the subject for treatment with a CDK inhibitor and a PIM inhibitor; and (c) administering a therapeutically effective amount of a CDK inhibitor and a PIM inhibitor to the subject. Specific embodiments of this aspect relate to detecting amplification, activation or overexpression PIM2 expression and treatment with a PIM2 inhibitor and a CDK4/6 inhibitor.

In another aspect, the invention provides a method of treating cancer in a subject in need thereof, comprising: (a) detecting the presence of PIM amplification, PIM activation and/or PIM overexpression in a biological sample from the subject; (b) selecting the subject for treatment with a combination comprising a CDK inhibitor and a PIM inhibitor; and (c) administering the combination to the subject. Specific embodiments of this aspect relate to detecting amplification, activation and/or overexpression PIM2 and selecting the subject for treatment with a combination comprising a CDK4/6 inhibitor and PIM2 inhibitor.

In another aspect, the invention provides a method of treating cancer in a subject in need thereof, comprising administering to the subject a therapeutically effective amount of a CDK inhibitor and a PIM inhibitor, wherein the subject has been identified as having PIM amplification, PIM activation and/or PIM overexpression. Specific embodiments of this aspect relate to treatment of subjects identified as having amplification, activation and/or overexpression of PIM2.

In another aspect, the invention provides a method of treating cancer in a subject in need thereof, comprising administering to the subject a combination comprising a CDK inhibitor and a PIM inhibitor, wherein the subject has been identified as having PIM amplification, PIM activation and/or PIM overexpression. Specific embodiments comprise administering a CDK4/6 inhibitor and a PIM2 inhibitor to a subject identified as having amplification, activation and/or overexpression of PIM2.

In another aspect, the invention provides a method of reversing, ameliorating or preventing decreased responsiveness to a CDK4/6 inhibitor in a subject having cancer, comprising: (a) obtaining a biological sample from the subject; (b) detecting the presence of PIM amplification, PIM activation and/or PIM overexpression in the biological sample; and (c) administering an effective amount of a CDK4/6 inhibitor and a PIM inhibitor to the subject. In some embodiments, such administration thereby reverses, ameliorates or prevents the decreased responsiveness to the CDK4/6 inhibitor in the subject. Specific embodiments comprise detecting PIM2 amplification, activation and/or overexpression and administering a CDK4/6 inhibitor and a PIM2 inhibitor to the subject.

In another aspect, the invention provides a method of reversing, ameliorating or preventing decreased responsiveness to a CDK4/6 inhibitor and endocrine therapy in a subject having cancer, comprising: (a) obtaining a biological sample from the subject; (b) detecting the presence of PIM amplification, PIM activation and/or PIM overexpression in the biological sample; and (c) administering an effective amount of a CDK4/6 inhibitor, a PIM inhibitor, and an endocrine therapeutic agent to the subject. In some embodiments, such administration thereby reverses or prevents the decreased responsiveness to the CDK4/6 inhibitor and endocrine therapy in the subject. Specific embodiments comprise detecting PIM2 amplification, activation and/or overexpression and administering a CDK4/6 inhibitor, a PIM2 inhibitor, and an endocrine therapeutic agent to the subject.

In another aspect, the invention provides a method of predicting efficacy of a CDK4/6 inhibitor and endocrine therapy in a subject having cancer, comprising: (a) obtaining a biological sample from the subject; and (b) detecting the presence of PIM amplification, PIM activation and/or PIM overexpression in the biological sample; wherein the presence of PIM amplification, PIM activation and/or PIM overexpression is predictive of decreased responsiveness to treatment. Specific embodiments comprise detecting PIM2 amplification, activation and/or overexpression in the sample, where such PIM2 amplification, activation and/or overexpression is predictive of decreased responsiveness to treatment.

In another aspect, the invention provides a method of predicting the efficacy of treatment with a CDK4/6 inhibitor and endocrine therapy in a subject having cancer, comprising detecting the presence or absence of PIM amplification, PIM activation and/or PIM overexpression in a biological sample from the subject, wherein the presence of PIM amplification, PIM activation and/or PIM overexpression is predictive of decreased responsiveness to treatment Specific embodiments comprise detecting the presence or absence of PIM2 amplification, activation and/or overexpression in the sample, wherein the presence such PIM2 amplification, activation and/or overexpression is predictive of decreased responsiveness to treatment. In some embodiments, the method further comprises treatment with a CDK4/6 inhibitor, endocrine therapy and a PIM inhibitor. In some such embodiments, the PIM inhibitor is a PIM2 inhibitor.

In another aspect, the invention provides a method of predicting the efficacy of a combination comprising a CDK4/6 inhibitor and endocrine therapeutic agent in a subject having cancer, comprising detecting the presence or absence of PIM amplification, PIM activation and/or PIM overexpression in a biological sample from the subject, wherein the presence of PIM amplification, PIM activation and/or PIM overexpression is predictive of decreased responsiveness to the combination. Specific embodiments comprise detecting the presence or absence of PIM2 amplification, activation and/or overexpression in the sample, wherein the presence such PIM2 amplification, activation and/or overexpression is predictive of decreased responsiveness to the combination. In some embodiments, the method further comprises treatment with a combination comprising a CDK4/6 inhibitor, endocrine therapeutic agent and a PIM inhibitor. In some such embodiments, the PIM inhibitor is a PIM2 inhibitor. In some embodiments of each of the aspects described herein related to methods of patient selection, the CDK inhibitor is an inhibitor of cyclin dependent kinase 4 (CDK4) and/or cyclin dependent kinase 6 (CDK6). In particular embodiments, the CDK inhibitor is a CDK4/6 inhibitor (i.e., an inhibitor of both CDK4 and CDK6). In other embodiments of each of the aspects described herein, the CDK inhibitor is a cyclin dependent kinase 4 (CDK4) inhibitor, a cyclin dependent kinase 6 (CDK6) inhibitor or a cyclin dependent kinase 4 and 6 (CDK4/6) inhibitor. In specific embodiments of each of the aspects described herein, the CDK inhibitor is a CDK4/6 inhibitor.

In preferred embodiments of each of the aspects described herein, the CDK4/6 inhibitor is palbociclib or a pharmaceutically acceptable salt thereof.

In other embodiments of each of the aspects described herein, the CDK4/6 inhibitor is ribociclib or abemaciclib or a pharmaceutically acceptable salt thereof. In some embodiments, the CDK4/6 inhibitor is ribociclib or a pharmaceutically acceptable salt thereof. In some embodiments, the CDK4/6 inhibitor is abemaciclib or a pharmaceutically acceptable salt thereof.

In some embodiments of each of the aspects herein, the PIM inhibitor is an inhibitor of one or more of proviral integration site for Moloney murine leukemia virus 1 (PIM1), proviral integration site for Moloney murine leukemia virus 2 (PIM2) or proviral integration site for Moloney murine leukemia virus 3 (PIM3). In some embodiments, the PIM inhibitor is a PIM2 inhibitor. In other embodiments, the PIM inhibitor is a PIM1 inhibitor. In some embodiments, the PIM inhibitor is a PIM1 and PIM2 inhibitor. In further embodiments, the PIM inhibitor is a PIM1, PIM2 and PIM3 inhibitor.

In another embodiment of each of the aspects herein, the CDK inhibitor is a CDK4/6 inhibitor and the PIM inhibitor is a PIM2 inhibitor.

In another embodiment of each of the aspects herein, the CDK inhibitor is palbociclib or a pharmaceutically acceptable salt thereof and the PIM inhibitor is a PIM2 inhibitor.

In some embodiments of the aspects herein, the PIM inhibitor is selected from the group consisting of AZD1208, PIM447 (LGH477), TP-3654, INCB053914 and LGB321, or a pharmaceutically acceptable salt thereof. In some embodiments, the PIM inhibitor is INCB053914, or a pharmaceutically acceptable salt thereof. In some embodiments, the PIM inhibitor is TP-3654, or a pharmaceutically acceptable salt thereof. In some embodiments, the PIM inhibitor is PIM447, or a pharmaceutically acceptable salt thereof.

In each of the aspects and embodiments described herein, the CDK inhibitor and the PIM inhibitor may independently optionally be in the form of a pharmaceutically acceptable salt.

In frequent embodiments of the invention, the subject is a human.

Examples of cancer in connection with the present invention include, but are not limited to, cancers of the breast, ovary, lung (including SCLC and NSCLC), skin, colon, bladder, liver, stomach, prostate, kidney, esophagus, nasopharynx, thyroid, cervix, pancreas, head and neck, or sarcomas, or a combination of one or more of the foregoing cancers. In some embodiments of the invention, the cancer is a solid tumor.

In frequent embodiments of each of the aspects described herein, the cancer is breast cancer. In some embodiments, the cancer is HR+ breast cancer (i.e., ER+ and/or PR+ breast cancer). In some embodiments, the cancer is ER+ breast cancer. In some embodiments, the cancer is PR+ breast cancer. In some embodiments, the cancer is ER+ and PR+ breast cancer.

In further embodiments, the cancer is HER2− breast cancer. In frequent embodiments, the cancer is HR+ HER2− breast cancer, ER+ HER2− breast cancer, or PR+ HER2− breast cancer.

In some embodiment of each of the aspects described herein, the cancer is locally advanced. In some embodiments of each of the aspects described herein, the cancer is metastatic. In other embodiments of each of the aspects described herein, the cancer is refractory.

In some embodiments of each of the aspects described herein, the cancer is resistant to treatment with a CDK inhibitor, e.g., a CDK4/6 inhibitor. In some such embodiments, the cancer is resistant to treatment with a CDK4/6 inhibitor in combination with endocrine therapy (e.g., an aromatase inhibitor or a SERD). In further embodiments of each of the aspects described herein, the cancer is resistant to treatment with endocrine therapy. In some such embodiments, the cancer is breast cancer that is resistant to treatment with endocrine therapy, such as aromatase inhibitors, SERDs or SERMs. In other embodiments, the cancer is resistant to treatment with chemotherapeutic agents, such as platinum agents, taxanes, docetaxel or gemcitabine.

In a further aspect, the invention provides a method of inhibiting cancer cell proliferation in a subject, comprising administering to the subject a combination therapy comprising a CDK inhibitor and a PIM inhibitor, in an amount effective to inhibit cell proliferation.

In another aspect, the invention provides a method of inhibiting cancer cell invasiveness in a subject, comprising administering to the subject a combination therapy which comprises a CDK inhibitor and a PIM inhibitor, in an amount effective to inhibit cell invasiveness.

In another aspect, the invention provides a method of inducing apoptosis in cancer cells in a subject, comprising administering to the subject a combination therapy which comprises a CDK inhibitor and a PIM inhibitor, in an amount effective to induce apoptosis.

### Dosage Forms and Regimens

Each therapeutic agent of the methods and combination therapies of the present invention may be administered either alone, or in a medicament (also referred to herein as a pharmaceutical composition) which comprises the therapeutic agent and one or more pharmaceutically acceptable carriers, excipients, or diluents, according to pharmaceutical practice.

As used herein, the terms “combination therapy” or “combination” refer to the administration or use of each therapeutic agent indicated as part of the combination therapy, alone (as single agents) or in a medicament, either sequentially, concurrently or simultaneously.

As used herein, the term “sequential” or “sequentially” refers to the administration of each therapeutic agent of the combination therapy of the invention, either alone or in a medicament, one after the other, wherein each therapeutic agent can be administered in any order. Sequential administration may be particularly useful when the therapeutic agents in the combination therapy are in different dosage forms, for example, one agent is a tablet and another agent is a sterile liquid, and/or the agents are administered according to different dosing schedules, for example, one agent is administered daily, and the second agent is administered less frequently such as weekly.

As used herein, the term “concurrently” refers to the administration of each therapeutic agent in the combination therapy of the invention, either alone or in separate medicaments, wherein the second therapeutic agent is administered immediately after the first therapeutic agent, but that the therapeutic agents can be administered in any order. In a preferred embodiment the therapeutic agents are administered concurrently.

As used herein, the term “simultaneous” refers to the administration of each therapeutic agent of the combination therapy of the invention in the same medicament.

In some embodiments of the present invention, the CDK inhibitor and the PIM inhibitor are administered sequentially, simultaneously or concurrently. In some such embodiments, the CDK inhibitor is administered before administration of the PIM inhibitor. In other embodiments, the CDK inhibitor is administered after administration of the PIM inhibitor. In other embodiments, the CDK inhibitor is administered concurrently with administration of the PIM inhibitor. In further embodiments, the CDK inhibitor is administered simultaneously with the PIM inhibitor. In some such embodiments of each of the foregoing, the CDK inhibitor is a CDK4/6 inhibitor. In other embodiments, the CDK inhibitor is a CDK4 inhibitor, a CDK6 inhibitor or a CDK4/6 inhibitor. In specific embodiments, the CDK inhibitor is palbociclib or a pharmaceutically acceptable salt thereof. Where an additional therapeutic agent, such as an endocrine therapeutic agent, is administered, it may be administered on the same or a different schedule from the CDK inhibitor and/or the PIM inhibitor.

As will be understood by those skilled in the art, the combination therapy may be usefully administered to a subject during different stages of their treatment.

In some embodiments, the combination therapy is administered to a subject who is previously untreated, i.e. is treatment naïve.

In some embodiments, the combination therapy is administered to a subject who has failed to achieve a sustained response after a prior therapy with a biotherapeutic or chemotherapeutic agent, i.e. is treatment experienced.

The combination therapy may be administered prior to of following surgery to remove a tumor and/or may be used prior to, during or after radiation therapy, and/or may be used prior to, during or after chemotherapy.

The efficacy of combinations described herein in certain tumors may be enhanced by combination with other approved or experimental cancer therapies, e.g., radiation, surgery, chemotherapeutic agents, targeted therapies, agents that inhibit other signaling pathways that are dysregulated in tumors, and other immune enhancing agents. The methods, combinations and uses of the current invention may further comprise one or more additional therapeutic agents. In some embodiments, the one or more additional therapeutic agents are selected from an aromatase inhibitor, a SERD, a SERM, an anti-androgen agent, a PI3K inhibitor, an mTOR inhibitor, a PARP inhibitor, a TGF-β inhibitor or an anti-PD axis antagonist (e.g., a PD-1 or PD-L1 antagonist).

In some embodiments of each of the aspects herein, the additional therapeutic agent is an endocrine therapeutic agent. In some such embodiments the endocrine therapeutic agent is selected from the group consisting of an aromatase inhibitor, a selective estrogen receptor degrader (SERD), and a selective estrogen receptor modulator (SERM). In some embodiments, the endocrine therapeutic agent is selected from the group consisting of letrozole, anastrozole, exemestane and fulvestrant.

In some embodiments of each of the methods described herein, the invention provides a method further comprising administering to the subject an amount of one or more additional therapeutic agents. In some such embodiments, the amounts of the CDK inhibitor, PIM inhibitor and additional therapeutic agent(s) are together effective to treat cancer. It will be understood that the CDK inhibitor, the PIM inhibitor, and the one or more additional therapeutic agents may be administered sequentially, concurrently or simultaneously.

Administration of combinations of the invention may be affected by any method that enables delivery of the compounds to the site of action. These methods include oral routes, intraduodenal routes, parenteral injection (including intravenous, subcutaneous, intramuscular, intravascular or infusion), topical, and rectal administration.

Dosage regimens may be adjusted to provide the optimum desired response. For example, a therapeutic agent of the combination therapy of the present invention may be administered as a single bolus, as several divided doses administered over time, or the dose may be proportionally reduced or increased as indicated by the exigencies of the therapeutic situation. It may be particularly advantageous to formulate a therapeutic agent in a dosage unit form for ease of administration and uniformity of dosage. Dosage unit form, as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention may be dictated by and directly dependent on (a) the unique characteristics of the chemotherapeutic agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose may be readily established, and the effective amount providing a detectable therapeutic benefit to a subject may also be determined, as can the temporal requirements for administering each agent to provide a detectable therapeutic benefit to the subject. Accordingly, while certain dose and administration regimens are exemplified herein, these examples in no way limit the dose and administration regimen that may be provided to a subject in practicing the present invention.

It is to be noted that dosage values may vary with the type and severity of the condition to be alleviated and may include single or multiple doses. It is to be further understood that for any particular subject, specific dosage regimens should be adjusted over time according to the individual need and the professional judgment of the person administering or supervising the administration of the compositions, taking into consideration factors such as the severity of the disorder or condition, the rate of administration, the disposition of the compound and the discretion of the prescribing physician. The dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan. Determining appropriate dosages and regimens for administration of the chemotherapeutic agent are well-known in the relevant art and would be understood to be encompassed by the skilled artisan once provided the teachings disclosed herein.

In some embodiments, at least one of the therapeutic agents in the combination therapy is administered using the same dosage regimen (dose, frequency and duration of treatment) that is typically employed when the agent is used as a monotherapy for treating the same cancer. In other embodiments, the subject received a lower total amount of at least one of the therapeutic agents in the combination therapy than when the same agent is used as a monotherapy, for example a lower dose of therapeutic agent, a reduced frequency of dosing and/or a shorter duration of dosing.

An effective dosage of a small molecule inhibitor is typically in the range of from about 0.001 to about 100 mg per kg body weight per day, preferably about 1 to about 35 mg/kg/day, in single or divided doses. For a 70 kg human, this would amount to about 0.01 to about 7 g/day, preferably about 0.02 to about 2.5 g/day, and more preferably from about 0.02 to about 1.0 g/day. In some instances, dosage levels at the lower limit of the aforesaid range may be more than adequate, while in other cases still larger doses may be employed without causing any harmful side effect, provided that such larger doses are first divided into several small doses for administration throughout the day.

In some embodiments, the CDK inhibitor, or a pharmaceutically acceptable salt or solvate thereof, is administered at a daily dosage of from about 50 mg to about 1000 mg per day, preferably from about 50 mg to about 600 mg per day, and more preferably from about 75 mg to about 200 mg per day. In certain embodiments, the CDK inhibitor is palbociclib, or a pharmaceutically acceptable salt or solvate thereof, which is administered at a daily dosage of 75 mg, 100 mg, or 125 mg per day. In other embodiments, the CDK inhibitor is ribociclib, or a pharmaceutically acceptable salt or solvate thereof, which is administered at a daily dosage of about 200 mg to about 600 mg per day; or abemaciclib, or a pharmaceutically acceptable salt or solvate thereof, which is administered at a daily dosage of about 150 mg or about 400 mg per day.

In some embodiments, the CDK inhibitor, or a pharmaceutically acceptable salt or solvate thereof, is administered at a daily dosage of about 50 mg, about 75 mg, about 100 mg, about 125 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1100 mg, about 1200 mg, about 1300 mg, about 1400 mg, or about 1500 mg. This dosage may be administered as a single dose (q.d.), or optionally may be subdivided into smaller doses, suitable for b.i.d., t.i.d. or q.i.d. administration.

In some embodiments, the PIM inhibitor, or a pharmaceutically acceptable salt or solvate thereof, is administered at a daily dosage of about 50 mg, about 75 mg, about 100 mg, about 125 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1100 mg, about 1200 mg, about 1300 mg, about 1400 mg, or about 1500 mg. This dosage may be administered as a single dose (q.d.), or optionally may be subdivided into smaller doses, suitable for b.i.d., t.i.d. or q.i.d. administration.

Repetition of the administration or dosing regimens, or adjustment of the administration or dosing regimen may be conducted as necessary to achieve the desired treatment. An “intermittent dosing schedule” as used herein refers to an administration or dosing regimen that includes a period of dose interruption, e.g. days off treatment. Repetition of 14 or 21 day treatment cycles with a 7 day treatment interruption between the treatment cycles is an example of an intermittent dosing schedule. Such schedules, with 2 or 3 weeks on treatment and 1 week off treatment, are sometimes referred to as a 2/1-week or 3/1-week treatment cycle, respectively.

A “continuous dosing schedule” as used herein is an administration or dosing regimen without dose interruptions, e.g. without days off treatment. Repetition of 21 or 28 day treatment cycles without dose interruptions between the treatment cycles is an example of a continuous dosing schedule.

In some embodiments, the CDK inhibitor and the PIM inhibitor are administered in an intermittent dosing schedule. In other embodiments, the CDK inhibitor and the PIM inhibitor are administered in a continuous dosing schedule.

In still other embodiments, one of the CDK inhibitor and the PIM inhibitor is administered in an intermittent dosing schedule (e.g., a 2/1-week or 3/1-week schedule) and the other is administered in a continuous dosing schedule. In some such embodiments, the CDK inhibitor is administered in an intermittent dosing schedule and the PIM inhibitor is administered in a continuous dosing schedule. In other such embodiments, the CDK inhibitor is administered in a continuous dosing schedule and the PIM inhibitor is administered in an intermittent dosing schedule.

In some embodiments of the present invention, the CDK inhibitor and the PIM inhibitor are dosed in amounts which together are effective in treating the cancer.

In some embodiments of the present invention, the CDK inhibitor and the PIM inhibitor are dosed in amounts which together are synergistic.

In some embodiments of the present invention, the CDK inhibitor and the PIM inhibitor are dosed in amounts which together are additive.

In each of the aspects and embodiments described herein, it will be understood that the CDK inhibitor may be a CDK4 inhibitor, a CDK6 inhibitor or a CDK4/6 inhibitor.

### Pharmaceutical Compositions and Routes of Administration

A “pharmaceutical composition” refers to a mixture of one or more of the therapeutic agents described herein, or a pharmaceutically acceptable salt, solvate, hydrate or prodrug thereof as an active ingredient, and at least one pharmaceutically acceptable carrier or excipient. In some embodiments, the pharmaceutical composition comprises two or more pharmaceutically acceptable carriers and/or excipients.

As used herein, a “pharmaceutically acceptable carrier” refers to a carrier or diluent that does not cause significant irritation to an organism and does not abrogate the biological activity and properties of the active compound or therapeutic agent.

The pharmaceutical acceptable carrier may comprise any conventional pharmaceutical carrier or excipient. The choice of carrier and/or excipient will to a large extent depend on factors such as the particular mode of administration, the effect of the excipient on solubility and stability, and the nature of the dosage form.

Suitable pharmaceutical carriers include inert diluents or fillers, water and various organic solvents (such as hydrates and solvates). The pharmaceutical compositions may, if desired, contain additional ingredients such as flavorings, binders, excipients and the like. Thus, for oral administration, tablets containing various excipients, such as citric acid may be employed together with various disintegrants such as starch, alginic acid and certain complex silicates and with binding agents such as sucrose, gelatin and acacia. Examples, without limitation, of excipients include calcium carbonate, calcium phosphate, various sugars and types of starch, cellulose derivatives, gelatin, vegetable oils and polyethylene glycols. Additionally, lubricating agents such as magnesium stearate, sodium lauryl sulfate and talc are often useful for tableting purposes. Solid compositions of a similar type may also be employed in soft and hard filled gelatin capsules. Non-limiting examples of materials, therefore, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

The pharmaceutical composition may, for example, be in a form suitable for oral administration as a tablet, capsule, pill, powder, sustained release formulation, solution or suspension, for parenteral injection as a sterile solution, suspension or emulsion, for topical administration as an ointment or cream, or for rectal administration as a suppository.

Exemplary parenteral administration forms include solutions or suspensions of an active compound in a sterile aqueous solution, for example, aqueous propylene glycol or dextrose solutions. Such dosage forms may be suitably buffered, if desired.

The pharmaceutical composition may be in unit dosage forms suitable for single administration of precise amounts.

Pharmaceutical compositions suitable for the delivery of the therapeutic agents of the combination therapies of the present invention, and methods for their preparation will be readily apparent to those skilled in the art. Such compositions and methods for their preparation may be found, for example, in ‘Remington's Pharmaceutical Sciences’, 19th Edition (Mack Publishing Company, 1995), the disclosure of which is incorporated herein by reference in its entirety.

Therapeutic agents of the combination therapies of the invention may be administered orally. Oral administration may involve swallowing, so that the therapeutic agent enters the gastrointestinal tract, or buccal or sublingual administration may be employed by which the therapeutic agent enters the blood stream directly from the mouth.

Formulations suitable for oral administration include solid formulations such as tablets, capsules containing particulates, liquids, or powders, lozenges (including liquid-filled), chews, multi- and nano-particulates, gels, solid solution, liposome, films (including muco-adhesive), ovules, sprays and liquid formulations.

Liquid formulations include suspensions, solutions, syrups and elixirs. Such formulations may be used as fillers in soft or hard capsules and typically include a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

Therapeutic agents of the combination therapies of the present invention may also be used in fast-dissolving, fast-disintegrating dosage forms such as those described in Expert Opinion in Therapeutic Patents, 11 (6), 981-986 by Liang and Chen (2001), the disclosure of which is incorporated herein by reference in its entirety.

For tablet dosage forms, the therapeutic agent may make up from 1 wt % to 80 wt % of the dosage form, more typically from 5 wt % to 60 wt % of the dosage form. In addition to the active agent, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinized starch and sodium alginate. Generally, the disintegrant may comprise from 1 wt % to 25 wt %, preferably from 5 wt % to 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinized starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally include surface active agents, such as sodium lauryl sulfate and polysorbate 80, and glidants such as silicon dioxide and talc. When present, surface active agents are typically in amounts of from 0.2 wt % to 5 wt % of the tablet, and glidants typically from 0.2 wt % to 1 wt % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally are present in amounts from 0.25 wt % to 10 wt %, preferably from 0.5 wt % to 3 wt % of the tablet.

Other conventional ingredients include anti-oxidants, colorants, flavoring agents, preservatives and taste-masking agents.

Exemplary tablets may contain up to about 80 wt % active agent, from about 10 wt % to about 90 wt % binder, from about 0 wt % to about 85 wt % diluent, from about 2 wt % to about 10 wt % disintegrant, and from about 0.25 wt % to about 10 wt % lubricant.

Tablet blends may be compressed directly or by roller to form tablets. Tablet blends or portions of blends may alternatively be wet-, dry-, or melt-granulated, melt congealed, or extruded before tableting. The final formulation may include one or more layers and may be coated or uncoated; or encapsulated.

The formulation of tablets is discussed in detail in “Pharmaceutical Dosage Forms: Tablets, Vol. 1”, by H. Lieberman and L. Lachman, Marcel Dekker, N.Y., N.Y., 1980 (ISBN 0-8247-6918-X), the disclosure of which is incorporated herein by reference in its entirety.

Solid formulations for oral administration may be formulated to be immediate and/or modified release. Modified release formulations include delayed-, sustained-, pulsed-, controlled-, targeted and programmed release.

Suitable modified release formulations are described in U.S. Pat. No. 6,106,864. Details of other suitable release technologies such as high energy dispersions and osmotic and coated particles may be found in Verma et al, Pharmaceutical Technology On-line, 25(2), 1-14 (2001). The use of chewing gum to achieve controlled release is described in WO 00/35298. The disclosures of these references are incorporated herein by reference in their entireties.

In one embodiment, a pharmaceutical composition useful for the combination therapy of the present invention comprises only a single therapeutic agent, for example either a CDK inhibitor or a PIM inhibitor.

In another embodiment, a pharmaceutical composition useful for the combination therapy of the present invention comprises both a CDK inhibitor and a PIM inhibitor.

The therapeutic agents of the combination therapies of the present invention may conveniently be combined in the form of a kit suitable for coadministration of the compositions.

In one aspect, the invention provides a kit which comprises a first container, a second container and a package insert, wherein the first container comprises at least one dose of a CDK inhibitor; the second container comprises at least one dose of a PIM inhibitor; and the package insert comprises instructions for treating cancer in a subject using the medicaments.

In one embodiment, the kit of the present invention may comprise one or both of the active agents in the form of a pharmaceutical composition, which pharmaceutical composition comprises an active agent, or a pharmaceutically acceptable salt or solvate thereof, and a pharmaceutically acceptable carrier. The kit may contain means for separately retaining said compositions, such as a container, divided bottle, or divided foil packet. An example of such a kit is the familiar blister pack used for the packaging of tablets, capsules and the like.

The kit may be particularly suitable for administering different dosage forms, for example, oral and parenteral, for administering the separate compositions at different dosage intervals, or for titrating the separate compositions against one another. To assist compliance, the kit typically includes directions for administration and may be provided with a memory aid. The kit may further comprise other materials that may be useful in administering the medicaments, such as diluents, filters, IV bags and lines, needles and syringes, and the like.

These and other aspects of the invention, including the exemplary specific embodiments listed below, will be apparent from the teachings contained herein.

### EXAMPLES

### Example 1—Gene Expression Analysis

Tumor samples were collected from subjects in two double-blind, phase 3 placebo-controlled studies that randomized subjects having ER+, HER2− (Study 1) or HR+, HER2− (Study 2) breast cancer 2:1.

In Study 1, postmenopausal women with ER+/HER2− advanced breast cancer (ABC) without prior systematic therapy for their advanced disease received either: (i) palbociclib (125 mg QD, 3/1 schedule) plus letrozole (2.5 mg QD) or (ii) placebo (3/1 schedule QD) plus letrozole (2.5 mg QD). The primary outcome was PFS as assessed by the investigator.

In Study 2, endocrine-pretreated HR+/HER2− advanced or metastatic breast cancer (MBC) patients received either: (i) palbociclib (125 mg QD, 3/1 schedule) plus fulvestrant (500 mg IM on Days 1 and 15 of Cycle 1, and then Day 1 of each subsequent 28-day cycle) or (ii) placebo (3/1 schedule QD) plus fulvestrant (500 mg IM on Days 1 and 15 of Cycle 1, and then Day 1 of each subsequent 28-day cycle). The primary outcome was PFS as assessed by the investigator.

Tumor samples were analyzed by EdgeSeq Oncology BM Panel (HTG Molecular Diagnostics; https://www.htgmolecular.com/assays/obp) for mRNA expression assessment of 2534 cancer-related genes. Cox regression analysis was performed to investigate potential interaction between biomarker levels, as either a continuous variable or dichotomized by median level, and treatment effect in terms of progression-free survival (PFS). Gene expression analysis was performed blinded to the clinical information.

The EdgeSeq system used was validated and used targeted capture sequencing to quantitate RNA expression levels of gene targets in formalin-fixed paraffin-embedded tumor tissues. Sample preparation was conducted by following the laboratory process and manufacturer protocols. Sequencing was performed on the Illumina NextSeq 500 Sequencer.

FIG. 1A shows PFS by expression levels of PIM2 (higher or lower based on dichotomization by median) in the Study 1 cohort. FIG. 1B shows PIM2 expression comparison in two treatment arms of the Study 1 cohort.

FIG. 2A shows PFS by expression levels of PIM2 (higher or lower based on dichotomization by median) in the Study 2 cohort. FIG. 2B shows PFS by expression levels of PIM2 in the Study 2 cohort after adjusting for known clinicopathologic factors (visceral metastases, site of tissue collection, baseline ECOG performance status, recurrence type, prior chemotherapy, prior tamoxifen, and prior AI).

The magnitude of relative palbociclib benefit was found to be smaller in patients with higher PIM2 mRNA expression in both the Study 2 (FIG. 2A; continuous interaction P=0.0150) and Study 1 (FIG. 1A; continuous interaction P=0.0785) cohorts, despite similar levels of expression between the two arms (FIG. 1B) and accounting for baseline clinicopathological characteristics (FIG. 2B).

### Example 2—Clinical Trial for the Combination of a CDK4/6 Inhibitor and a PIM1 and/or PIM2 Inhibitor, Optionally in Further Combination with Letrozole

A non-limiting example of a breast cancer clinical trial in humans involving the combination of palbociclib or a pharmaceutically acceptable salt thereof and a PIM1 and/or PIM2 Inhibitor, optionally in further combination with letrozole, is described below.

Purpose: The purposes of this study is to assess the safety and efficacy of a combination of palbociclib or a pharmaceutically acceptable salt thereof and a PIM1 and/or PIM2 Inhibitor, optionally in further combination with letrozole, in humans with breast cancer (e.g., advanced or metastatic ER+/HR+ HER2− breast cancer), collect information on any side effects the combination therapy may cause, and evaluate the pharmacokinetic properties of the compounds in the context of combination therapy.

Intervention: Patients are administered palbociclib at a dose of 75 mg, 100 mg or 125 mg per day, on a 3/1 dosing schedule or continuously. Dose adjustment will be considered during the trial if deemed necessary. Patients will be administered a PIM1 and/or PIM2 inhibitor as a second therapeutic agent. For example, the PIM inhibitor INCB053914 or PIM447 is administered at an appropriate dose to be determined in a dose ranging study. Dose adjustment will be considered during the trial if deemed necessary. Optionally, the study will include an arm where the patients will be administered endocrine therapy as a third therapeutic agent, such as letrozole administered orally at a dose of about 2.5 mg QD.

Outcome Measures: PFS (Progression-free survival); overall survival (OS); objective response (OR); duration of response (DR); clinical benefit response (CBR); Survival Probabilities at Months 12, 24 and 36; quality of life (QOL); side-effects; pharmacokinetics (PK); tumor response and/or disease control; proportion of patients that have complete or partial response or stable disease at defined time points; biomarkers predictive of clinical response.

Eligibility: 18 years and older

Inclusion Criteria: Adult women (>18) with locoregionally recurrent or metastatic disease not amenable to curative therapy; confirmed diagnosis of ER positive breast cancer; no prior systemic anti-cancer therapy for advanced ER+ disease; postmenopausal women; measurable disease as per Response Evaluation Criterion in Solid Tumors [RECIST] or bone-only disease; Eastern Cooperative Oncology Group [ECOG] 0-2; Adequate organ and marrow function; patient must agree to provide tumor tissue.

Exclusion Criteria: Confirmed diagnosis of HER2-positive disease; patients with advanced, symptomatic, visceral spread that are at risk of life-threatening complication in the short term; Known uncontrolled or symptomatic CNS metastases; prior (neo)adjuvant treatment with letrozole or anastrozole with DFI≤12-months from completion of treatment; prior treatment with any CDK 4/6 inhibitor.

### Example 3—Clinical Trial for the Combination of a CDK4/6 Inhibitor and a PIM1 and/or PIM2 Inhibitor, Optionally in Further Combination with Fulvestrant

A non-limiting example of a breast cancer clinical trial in humans involving the combination of palbociclib or a pharmaceutically acceptable salt thereof and a PIM1 and/or PIM2 Inhibitor, optionally in further combination with fulvestrant, is described below.

Purpose: The purposes of this study is to assess the safety and efficacy of a combination of palbociclib or a pharmaceutically acceptable salt thereof and a PIM1 and/or PIM2 Inhibitor, optionally in further combination with fulvestrant, in humans with breast cancer (e.g., advanced or metastatic HR+ HER2− breast cancer), collect information on any side effects the combination therapy may cause, and evaluate the pharmacokinetic properties of the compounds in the context of combination therapy.

Intervention: Patients are administered palbociclib at a dose of 75 mg, 100 mg or 125 mg per day, on a 3/1 dosing schedule or continuously. Dose adjustment will be considered during the trial if deemed necessary. Patients will be administered a PIM1 and/or PIM2 inhibitor as a second therapeutic agent. for example, the PIM inhibitor INCB053914 or PIM447 is administered at an appropriate dose to be determined in a dose ranging study. Dose adjustment will be considered during the trial if deemed necessary. Optionally, the study will include an arm where the patients will be administered endocrine therapy as a third therapeutic agent, such as fulvestrant administered intramuscularly at a dose of about 500 mg on Days 1 and 15 of Cycle 1, and then on Day 1 of each subsequent 28 day cycle.

Outcome Measures: PFS (Progression-free survival); overall survival (OS); objective response (OR); duration of response (DR); clinical benefit response (CBR); Survival Probabilities at Months 12, 24 and 36; quality of life (QOL); side-effects; pharmacokinetics (PK); tumor response and/or disease control; proportion of patients that have complete or partial response or stable disease at defined time points; biomarkers predictive of clinical response.

Eligibility: 18 years and older Inclusion Criteria: Women 18 years or older with metastatic or locally advanced disease, not amenable to curative therapy; confirmed diagnosis of HR+/HER2− breast cancer; any menopausal status; progressed within 12 months from prior adjuvant or progressed within 1 month from prior advanced/metastatic endocrine breast cancer therapy; on an LHRH agonist for at least 28 days, if pre-/peri-menopausal, and willing to switch to goserelin (Zoladex®) at time of randomization; measurable disease defined by RECIST version 1.1, or bone-only disease; Eastern Cooperative Oncology Group (ECOG) PS 0-1; adequate organ and marrow function, resolution of all toxic effects of prior therapy or surgical procedures; patient must agree to provide tumor tissue from metastatic tissue at baseline.

Exclusion Criteria: Prior treatment with any CDK inhibitor, fulvestrant, everolimus, or agent that inhibits the PI3K-mTOR pathway; patients with extensive advanced/metastatic, symptomatic visceral disease, or known uncontrolled or symptomatic CNS metastases; major surgery or any anti-cancer therapy within 2 weeks of randomization; prior stem cell or bone marrow transplantation; use of potent CYP3A4 inhibitors or inducers.

### Example 4—Therapeutic Treatment

A patient having HR+/HER2− breast cancer is treated with a combination of palbociclib at a dose of 125 mg per day (3/1 dosing schedule) plus a PIM inhibitor, such as INCB053914 or PIM447, at an effective dose, thereby treating the cancer.

### Example 5—Therapeutic Treatment

A patient having HR+/HER2− breast cancer is treated with a combination of palbociclib (125 mg QD, 3/1 schedule), letrozole (2.5 mg QD) and a PIM inhibitor, such as INCB053914 or PIM447, at an effective dose, thereby treating the cancer.

### Example 6—Therapeutic Treatment

A patient having HR+/HER2− breast cancer is identified as having a cancer associated with PIM2 amplification, PIM2 activation and/or PIM2 overexpression. The patient is treated with a combination of palbociclib (125 mg QD, 3/1 schedule), letrozole (2.5 mg QD) and a PIM2 inhibitor, such as INCB053914 or PIM447, at an effective dose, thereby treating the cancer.

### Example 7—Patient Selection

The presence of PIM2 amplification, PIM2 activation and/or PIM2 overexpression is detected in a biological sample from a patient having HR+/HER2− breast cancer. The patient is selected for treatment and then treated with a combination of palbociclib (125 mg QD, 3/1 schedule), letrozole (2.5 mg QD) and a PIM2 inhibitor, such as INCB053914 or PIM447, at an effective dose, thereby treating the cancer.

All publications and patent applications cited in the specification are herein incorporated by reference in their entirety. Although the foregoing invention has been described in some detail by way of illustration and example, it will be readily apparent to those of ordinary skill in the art in light of the teachings of this invention that certain changes and modifications may be made thereto without departing from the spirit or scope of the appended claims.

1. A method of treating hormone receptor-positive (HR+), human epidermal growth factor receptor 2-negative (HER2−) breast cancer in a subject in need thereof, comprising administering to the subject a cyclin dependent kinase 4/6 (CDK4/6) inhibitor, a proviral integration site for Moloney murine leukemia virus 2 (PIM2) inhibitor, and an endocrine therapeutic agent, wherein the amounts of the CDK4/6 inhibitor, the PIM2 inhibitor, and the endocrine therapeutic agent are together effective to treat the cancer.

2. The method of claim 1, wherein the breast cancer is advanced or metastatic breast cancer, or early breast cancer.

3. The method of claim 1, wherein the cancer is associated with PIM2 amplification, PIM2 activation and/or PIM2 overexpression.

4. The method of claim 1, wherein the endocrine therapeutic agent is selected from the group consisting of an aromatase inhibitor, a selective estrogen receptor degrader (SERD), and a selective estrogen receptor modulator (SERM).

5. The method of claim 1, wherein the endocrine therapeutic agent is selected from the group consisting of letrozole, anastrozole, exemestane and fulvestrant.

6. The method of claim 1, wherein the CDK4/6 inhibitor is palbociclib, or a pharmaceutically acceptable salt thereof.

7. The method of claim 1, wherein the CDK4/6 inhibitor and the PIM2 inhibitor are administered sequentially, simultaneously or concurrently.

8. The method of claim 1, wherein the subject is identified as having PIM2 amplification, PIM2 activation and/or PIM2 overexpression.

9-15. (canceled)

16. A method of selecting a subject having cancer for treatment with a combination of a CDK4/6 inhibitor, a PIM2 inhibitor, and optionally an endocrine therapeutic agent, comprising: (a) detecting the presence of PIM2 amplification, PIM2 activation and/or PIM2 overexpression in a biological sample from the subject; and (b) selecting the subject for treatment with the combination of a CDK4/6 inhibitor, a PIM2 inhibitor, and optionally an endocrine therapeutic agent.

17. The method of claim 16, further comprising (c) administering an effective amount of a CDK4/6 inhibitor, a PIM2 inhibitor, and optionally an endocrine therapeutic agent to the subject.

18. A method of predicting whether a subject having cancer will be resistant to treatment with a CDK4/6 inhibitor and an endocrine therapeutic agent, comprising comparing the level of PIM2 expression in a biological sample from the subject to the level of PIM2 expression in a control sample, wherein increased PIM2 expression in the subject sample relative to the control sample indicates the subject is likely to be resistant to treatment with the CDK4/6 inhibitor and the endocrine therapeutic agent.

19. The method of claim 18, further comprising administering an effective amount of a CDK4/6 inhibitor, an endocrine therapeutic agent, and a PIM2 inhibitor to the subject.

20. A method of treating cancer in a subject, comprising: (a) detecting the presence of PIM2 amplification, PIM2 activation and/or PIM2 overexpression in a biological sample from the subject; (b) selecting the subject for treatment with a CDK4/6 inhibitor, a PIM2 inhibitor, and optionally an endocrine therapeutic agent; and (c) administering a therapeutically effective amount of a CDK4/6 inhibitor, a PIM2 inhibitor, and optionally an endocrine therapeutic agent, to the subject.
